(19) United States
(12) Patent Application Publication
(10) Pub. No.: US 2023/0106587 A1
(43) Pub. Date: Apr. 6, 2023

(54) CATHETER

(71) Applicant: TERUMO KABUSHIKI KAISHA, Tokyo (JP)

(72) Inventors: Takashi ITO, Fujinomiya-shi (JP); Keiichiro YAMAMOTO, Elkton, MD (US)

(73) Assignee: TERUMO KABUSHIKI KAISHA, Tokyo (JP)

(21) Appl. No.: 18/060,696

(22) Filed: Dec. 1, 2022

Related U.S. Application Data
(63) Continuation of application No. PCT/JP2021/012980, filed on Mar. 26, 2021.

(30) Foreign Application Priority Data
Jun. 12, 2020 (JP) 2020-102078

(51) Int. Cl. A61M 25/01

(52) U.S. Cl. CPC A61M 25/0108; A61M 25/0144; A61M 2025/1056

(57) ABSTRACT

A catheter includes a shaft having a lumen extending from a distal end to a proximal end. The shaft includes a reinforcement body including a plurality of wires that are disposed on at least a part between an inner surface of the shaft forming the lumen and an outer surface of the shaft and that are braided in a tubular shape, and at least one radiopaque marker disposed between the reinforcement body and the outer surface of the shaft. An outer diameter difference, which is a difference between an outer diameter of the shaft in a portion where the marker is disposed and an outer diameter of the shaft in a portion adjacent to the marker in an axial direction of the shaft, is smaller than twice a thickness of the marker.

### CROSS-REFERENCES TO RELATED APPLICATIONS

This application is a continuation of International Patent Application No. PCT/JP2021/012980 filed on Mar. 26, 2021, which claims priority to Japanese Patent Application No. 2020-102078 filed on Jun. 12, 2020, the entire content of both of which is incorporated herein by reference.

### TECHNOLOGICAL FIELD

The present invention generally relates to a catheter used in a lumen such as a blood vessel.

### BACKGROUND DISCUSSION

In recent years, for a reason that surgical invasiveness is fairly low, a treatment in a lumen such as a blood vessel using a catheter has been actively performed. For example, in general, the catheter used by being selectively introduced into a complexly branched blood vessel in a body is selectively pushed in along a guide wire introduced in advance into the blood vessel to circulate a therapeutic drug, a diagnostic contrast agent, or the like from a proximal side to a distal side.

A radiopaque marker is disposed in a shaft such that a position of the catheter in the lumen can be recognized from the outside of the body. For example, Japanese Patent Application Publication No. 2000-237319 (JP-A-2000-237319) describes a catheter in which a marker is fixed to an outer peripheral surface of a shaft by punching. Japanese Patent Application Publication No. 6-197974 (JP-A-6-197974) describes a catheter in which the marker is disposed on an inner peripheral surface of a shaft and from which a portion protruding in a radial direction on an outer surface of the shaft is removed such that the catheter surface is flat or smooth.

The shaft is formed thin such that the shaft can be inserted into the blood vessel or the like. Alternatively, in order to ensure pushing performance (pushability), torque transmission performance, or the like, a reinforcement body may be provided, and this reinforcement may be a braided wire. Meanwhile, for example, when the shaft is inserted from a radial artery of a hand and treatment of a lower limb is performed, a long shaft is required. When the shaft is long, it is difficult to transmit a pushing force and torque applied to a proximal portion of the shaft to a distal portion of the shaft. To improve the pushing performance and the torque transmission performance, for example, it is conceivable to increase a wire diameter of the wire of the reinforcement body.

### SUMMARY

When the wire diameter of the braided wire is increased, a thickness of the shaft of the portion where the marker is located is also increased when the marker is disposed in the shaft where the reinforcement body is embedded.

The catheter disclosed here is capable of preventing an increase in a thickness of a shaft while disposing a radiopaque marker on the shaft provided with a reinforcement body.

According to one aspect, the catheter includes a shaft having a lumen extending from a distal end to a proximal end of the shaft. The shaft includes a reinforcement body including a plurality of wires that are disposed on at least a part between an inner surface of the shaft forming the lumen and an outer surface of the shaft and that are braided in a tubular shape. The shaft additionally includes at least one radiopaque marker disposed between the reinforcement body and the outer surface. A difference between an outer diameter of the shaft in a portion where the marker is disposed and an outer diameter of the shaft in a portion adjacent to the marker in an axial direction of the shaft is defined as an outer diameter difference, and a difference between an inner diameter of the shaft in the portion where the marker is disposed and an inner diameter of the shaft in the portion adjacent to the marker in the axial direction of the shaft is defined as an inner diameter difference. At least one of the outer diameter difference and the inner diameter difference is smaller than twice a thickness of the marker.

In another aspect, the catheter includes a shaft having a lumen extending from a distal end to a proximal end. The shaft includes a reinforcement body including a plurality of wires that are disposed on at least a part between an inner surface of the shaft forming the lumen and an outer surface of the shaft and that are braided in a tubular shape. The shaft also includes at least one radiopaque marker disposed between the reinforcement body and the outer surface. A difference between a maximum outer diameter of the marker and a maximum outer diameter of the reinforcement body in a portion adjacent to the marker in an axial direction of the shaft is smaller than twice a thickness of the marker.

According to a further aspect, a catheter comprises a shaft possessing an open distal end and an open proximal end, with the shaft including a lumen surrounded by an inner surface of the shaft, and the lumen extending from the open distal end of the shaft to the open proximal end of the shaft. The shaft comprises a reinforcement body including a plurality of wires braided in a tubular shape so that the reinforcement body is a tubular reinforcement body possessing opposite axial ends. The tubular reinforcement body is located radially between the inner surface of the shaft and the outer surface of the shaft, and the tubular reinforcement body includes an axially extending smaller diameter portion positioned axially between two axially extending portions of the tubular reinforcement body that are immediately axially adjacent the small diameter portion, and the outer diameter of the smaller diameter portion of the tubular reinforcement body is less than the outer diameter of the two axially extending portions of the tubular reinforcement body that are immediately axially adjacent the smaller diameter portion. At least one radiopaque marker is disposed radially between the reinforcement body and the outer surface of the shaft, and the radiopaque marker is located in axially overlapping relation to the smaller diameter portion of the tubular reinforcement body. A difference between the outer diameter of the shaft in a portion where the radiopaque marker is disposed and the outer diameter of the shaft in a portion axially adjacent to the radiopaque marker in an axial direction of the shaft constitutes an outer diameter difference, and a difference between the inner diameter of the shaft in the portion axially adjacent to the radiopaque marker in the axial direction of the shaft and the inner diameter of the shaft in the portion where the radiopaque marker is disposed constitutes an inner diameter difference. At least one of the outer diameter difference and the inner diameter difference is smaller than twice the thickness of the radiopaque marker, or a difference between a maximum outer diameter of the radiopaque marker and a maximum outer diameter of the reinforcement body in a portion axially adjacent to the radiopaque marker in an axial direction of the shaft is smaller than twice a thickness of the radiopaque marker.

In the catheter configured as described above, the outer diameter of the shaft provided with the reinforcement body and the radiopaque marker is not too large, and/or the inner diameter of the shaft is not too small. Therefore, it is possible to prevent an increase in the thickness of the shaft while disposing the marker on the shaft provided with the reinforcement body.

A thickness of an outer layer of the shaft in the portion where the marker is disposed may be the same as a thickness of an outer layer of the shaft in the portion adjacent to the marker in the axial direction of the shaft. Accordingly, it is possible to prevent an increase in the thickness of the shaft while making the thickness of the outer layer uniform regardless of the presence or absence of the marker.

The marker may include at least one convex portion on at least an inner surface side. Accordingly, the marker is firmly fixed to the shaft. Therefore, detachment of the marker from the shaft can be prevented.

An effective length of the shaft may be 2100 mm or more. Accordingly, the catheter can easily reach an artery of a lower limb from an artery of an arm.

### BRIEF DESCRIPTION OF THE DRAWINGS

FIG. 1 is a plan view showing a catheter according to an embodiment.

FIG. 2 is a cross-sectional view showing a distal portion of the catheter according to the embodiment.

FIGS. 3A and 3B are cross-sectional views showing the distal portion of the catheter according to the embodiment, with FIG. 3A being a cross-sectional view taken along the section line 3A-3A in FIG. 2, and FIG. 3B is a cross-sectional view taken along the section line 3B-3B in FIG. 2.

FIG. 4 is a plan view showing the catheter through an outer layer of the catheter according to the embodiment.

FIG. 5 is a plan view showing a modification of the catheter.

FIG. 6 is a plan view showing another modification of the catheter.

### DETAILED DESCRIPTION

Set forth below with reference to the accompanying drawings is a detailed description of an embodiment of a catheter representing an example of the new catheter disclosed here. The dimensions or scales on the drawings may be exaggerated or different from actuality/reality for convenience of description and illustration. In the following description, a side on which a catheter is operated is referred to as a “proximal side”, and a side to be inserted into a living body is referred to as a “distal side”.

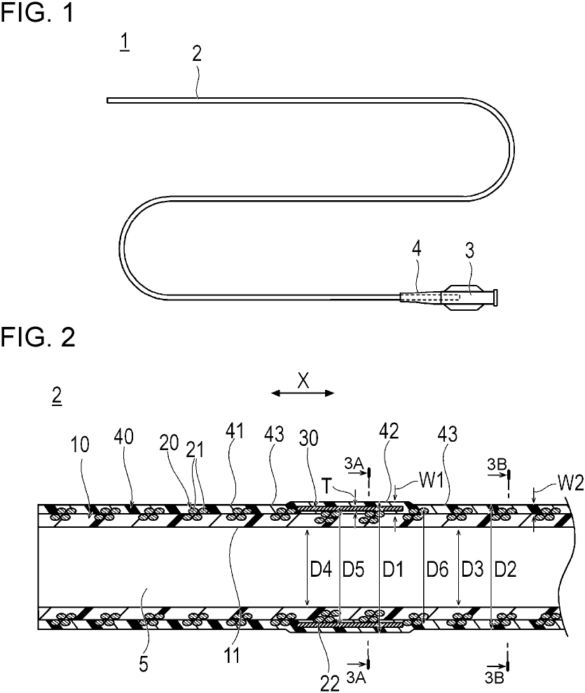

A catheter 1 according to the present embodiment is introduced into a blood vessel from a radial artery of an arm, inserted into an artery of a lower limb, and used for treatment, diagnosis, and the like. The artery of the lower limb is an artery near and more peripheral to an aortoiliac bifurcated portion. As shown in FIG. 1, the catheter 1 includes a long or elongated shaft 2, a hub 3 coupled to a proximal end of the shaft 2, and a strain relief 4 provided at a connection portion between the shaft 2 and the hub 3.

As shown in FIGS. 1 to 4, the shaft 2 is a flexible tubular member, and a lumen 5 is formed of located inside the shaft 2 from the proximal end to a distal end. A guide wire is inserted into the lumen 5 when the catheter 1 is inserted into a blood vessel. The lumen 5 can also be used as a passage for a drug solution, an embolic substance, a contrast agent, a medical instrument, or the like.

An effective length of the shaft 2 (i.e., the length from the distal end of shaft to the distal end of the strain relief or the maximum length of the catheter that can be inserted in the blood vessel) is not particularly limited, but is preferably 1500 mm to 2600 mm, more preferably 1800 mm to 2300 mm, still more preferably 2100 mm to 2300 mm, and is 2100 mm in the present embodiment. Accordingly, the catheter 1 can reach the artery of the lower limb from the artery of the arm. The effective length of the shaft 2 is a length of a portion that can be inserted into a blood vessel, a sheath, or the like. In the present embodiment, the effective length is a length from a distal-most end of the strain relief 4 to a distal-most end of the shaft 2. The effective length of the shaft 2 is preferably 650 mm or more when the catheter is introduced from a femoral artery, and is preferably 300 mm or more when the catheter approaches from a distal portion of a dorsalis pedis artery or a posterior tibial artery.

The shaft 2 is comprised of a plurality of layers, and includes an inner layer 10 forming an inner surface 11 of the lumen 5, a reinforcement body 20 formed on an outer side of the inner layer 10, a marker 30 disposed on an outer side of the reinforcement body 20 in a radial direction (i.e., the marker 30 is located radially outward of the reinforcement body 20), and an outer layer 40 formed on an outer side of the inner layer 10 and the reinforcement body 20. A thickness of the shaft 2 is larger or greater than the sum of a thickness of the reinforcement body 20 and a thickness of the marker 30. Therefore, the reinforcement body 20 and the marker 30 can be completely embedded inside the shaft 2.

An inner diameter of the inner layer 10 is not particularly limited, but is preferably 0.4 mm to 1.2 mm, more preferably 0.5 mm to 1.2 mm, still more preferably 0.95 mm to 1.2 mm, and is 1.05 mm in the present embodiment. The inner diameter of the inner layer 10 may be smaller than a portion adjacent in an axial direction X of the shaft 2 at a position covered by the marker 30.

The lumen 5 is formed inside the inner layer 10 so that the lumen 5 is radially inwardly of the inner layer 10. As a constituent material from which the inner layer 10 may be fabricated, a thermoplastic resin, a thermosetting resin, or the like can be used, and a fluorine-based resin such as polytetrafluoroethylene (PTFE), a low-friction material such as high-density polyethylene (HDPE), or the like is preferable.

The reinforcement body 20 is formed by braiding a plurality of wires 21 in a tubular shape around an outer periphery of the inner layer 10, and the wires 21 are braided in the tubular shape with gaps between adjacent windings. In the reinforcement body 20, a small diameter portion 22 having a locally small outer diameter is formed in order to dispose the marker 30. That is, an axially extending portion of the reinforcement body 20 is a smaller outer diameter portion having a smaller outer diameter than axially adjacent portions of the reinforcement body 20 that are immediately axially adjacent the small diameter portion 22, and the radiopaque marker is located at this smaller outer diameter portion.

A length of the small diameter portion 22 in the axial direction X is preferably larger than or equal to a length of the marker 30 in the axial direction X such that the marker 30 can be accommodated (the radiopaque marker 30 is located in axially overlapping relation to the small diameter portion 22 of the reinforcement body 20). The reinforcement body 20 may be formed by winding the wires 21 in horizontal winding in the same direction, or winding the wires 21 while changing a winding direction, such as right-handed winding and left-handed winding. A winding pitch, an interstitial distance, an inclination angle with respect to a peripheral direction, and the like may be changed depending on a position. The configuration is not particularly limited. The reinforcement body 20 may be configured so that the small diameter portion 22 is not included.

A difference (D5-D6) between a maximum outer diameter D5 of the marker 30 and a maximum outer diameter D6 of the reinforcement body 20 in the portion adjacent to the marker 30 in the axial direction X of the shaft 2 is preferably smaller than twice a thickness T of the marker 30.

A wire diameter (diameter) of the wire 21 of the reinforcement body 20 is not particularly limited, but is preferably 0.03 mm to 0.08 mm, more preferably 0.03 mm to 0.06 mm, still more preferably 0.04 mm to 0.06 mm, and is 0.05 mm in the present embodiment.

As the wire 21 used for the reinforcement body 20, a metal wire made of metal such as stainless steel, platinum (Pt) and tungsten (W), resin fiber, carbon fiber, glass fiber, or the like can be used, or the plurality of these wires 21 may be used in combination.

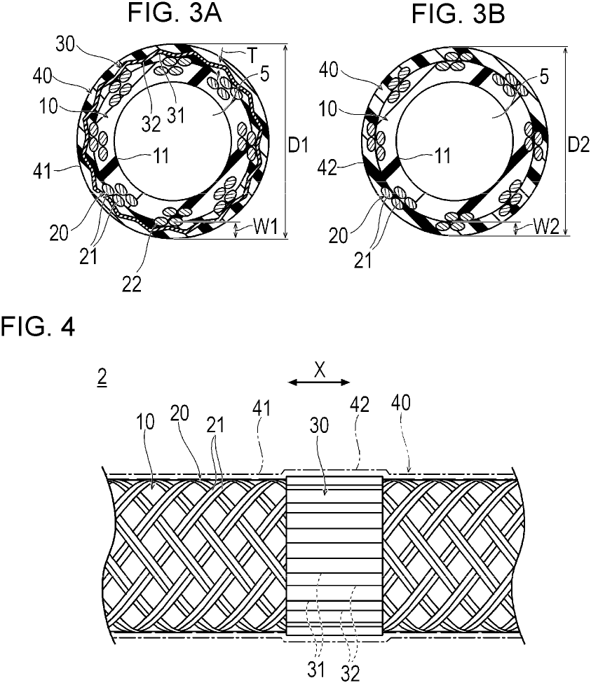

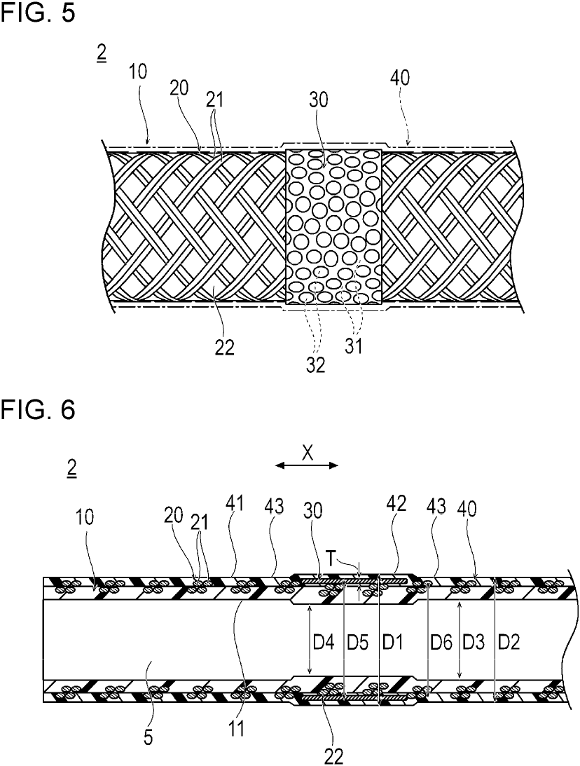

The marker 30 is a radiopaque (X-ray contrast) tube (tubular) body that enters or is positioned on the small diameter portion 22 from the outer side in the radial direction. When the marker 30 is disposed, after the reinforcement body 20 is disposed around the outer periphery of the inner layer 10, a tubular body formed of a material containing radiopaque substance is disposed in a manner of surrounding the inner layer 10 and the reinforcement body 20. Thereafter, the tubular body is crimped from the outer side in the radial direction to form a plurality of concave portions 31 and a plurality of convex portions 32 on an inner peripheral surface of the tubular body, and the tubular body is reduced in diameter to dispose the marker 30 in the shaft 2. As shown in FIGS. 3A and 4, the concave portions 31 and convex portions 32 alternate with one another in the circumferential direction of the tubular body constituting the marker 30. Therefore, a cross-sectional shape of the marker 30 in a cross-section orthogonal to the axial direction X (central axis) of the shaft 2 is non-circular. In the present embodiment, the concave portions 31 extend in the axial direction X of the shaft 2 and are formed in a groove shape (i.e., the concave portions 31 are groove-shaped or trough-shaped). The convex portions 32 extend in the axial direction X of the shaft 2 and are formed in a beam shape (i.e., the convex portions or ridges are beam-shaped or ridge-shaped). The concave portions 31 and the convex portions 32 are arranged substantially uniformly in the peripheral direction of the marker 30. The number of the concave portions 31 and the number of the convex portions 32 are not particularly limited, but a cross-sectional shape of the marker 30 approaches a circular shape as the number increases. The concave portions 31 may not be in the groove shape. The convex portions 32 may not be in the beam shape. For example, as in a modification shown in FIG. 5, the concave portions 31 and the convex portions 32 need not extend axially across the width of the tubular marker 30, and the concave portions 31 and the convex portions 32 may be randomly formed. As shown in FIG. 5, the concave portions 31 (depressions) and the convex portions 32 (protuberances) may be spaced apart from one another in both the width and circumferential directions

As shown in FIGS. 2, 3A and 3B, the thickness T of the marker 30 can be an average value of the thicknesses T of the marker 30 in the cross-section orthogonal to the central axis of the shaft 2 at a predetermined position along the marker 30 (for example, a center or an end portion in the axial direction X). The average value of the thicknesses T of the marker 30 in the cross-section orthogonal to the central axis of the shaft 2 may be, for example, a value obtained by dividing a cross-sectional area of the marker 30 in the cross-section by a length (for example, an average value of a peripheral length of the inner peripheral surface of the marker 30 and a peripheral length of the outer peripheral surface of the marker 30) of the marker 30 in the peripheral direction.

The thickness T of the marker 30 is not particularly limited, but is preferably 0.02 mm to 0.1 mm, more preferably 0.03 mm to 0.08 mm, still more preferably 0.03 mm to 0.04 mm, and is 0.03 mm in the present embodiment.

As a constituent material from which the marker 30 may be fabricated, a material kneaded with an X-ray contrast agent such as platinum, gold, silver, tungsten, iridium, or metal powders of an alloy thereof, or barium sulfate, bismuth oxide, or a coupling compound thereof can be suitably used.

As shown in FIGS. 2 to 4, the outer layer 40 is a tubular member that covers the outer periphery of the inner layer 10, the outer periphery of the reinforcement body 20, and the outer periphery of the marker 30. The outer layer 40 forms an outer surface 41 that is a surface on the outer side of the shaft 2 in the radial direction. The outer surface 41 includes an outer layer convex portion 42 formed at a position where the marker 30 is covered. As shown in FIG. 2, the outer layer convex portion 42 is a bulge that bulges radially outwardly relative to portions of the outer layer 40 on opposite axial ends of the bulge. An outer diameter D1 of the outer layer convex portion 42 is larger than an outer diameter D2 of the portion of the outer layer 40 adjacent to the outer layer convex portion 42 in the axial direction X of the shaft 2. That is, the outer diameter D1 of the outer layer convex portion 42 is greater than the outer diameter D2 of the portions of the outer layer 40 on opposite axial ends of the outer layer convex portion 42 An outer diameter difference (D1-D2), which is a difference between the outer diameter D1 of the outer layer convex portion 42 and the outer diameter D2 of the portion (adjacent portion 43) adjacent to the outer layer convex portion 42 in the axial direction X, is smaller than twice the thickness T of the marker 30.

The outer diameter D1 of the outer layer convex portion 42 is not particularly limited, but is preferably 0.7 mm to 1.6 mm, more preferably 1.3 mm to 1.5 mm, still more preferably 1.36 mm to 1.46 mm, and is 1.41 mm in the present embodiment.

The outer diameter D2 of the portion adjacent to the outer layer convex portion 42 is not particularly limited, but is preferably 0.7 mm to 1.6 mm, more preferably 1.3 mm to 1.5 mm, still more preferably 1.35 mm to 1.45 mm, and is 1.40 mm in the present embodiment. The outer diameter difference (D1-D2), which is the difference between the outer diameter D1 and the outer diameter D2, is preferably 0 mm to 0.06 mm, more preferably 0 mm to 0.03 mm, still more preferably 0 mm to 0.01 mm, and is 0.01 mm in the present embodiment.

As a constituent material from which the outer layer 40 may be fabricated, for example, a thermoplastic resin such as a polymer material such as polyolefin (for example, polyethylene, polypropylene, polybutene, an ethylene-propylene copolymer, an ethylene-vinyl acetate copolymer, an ionomer, or a mixture of two or more thereof), polyvinyl chloride, polyamide, a polyester elastomer, a polyamide elastomer, polyurethane, a polyurethane elastomer, polyimide, or fluororesin, or a mixture thereof, and a thermosetting resin such as epoxy resin can be used. The radiopaque substance may be mixed in the outer layer 40.

The proximal portion of the shaft 2 is fixed to the hub 3 in a liquid-tight manner by an adhesive, heat-welding, a fastener or the like. The hub 3 functions as an insertion port for a guide wire or a medical instrument into the lumen 5, an injection port for a drug solution, an embolic substance, a contrast agent, or the like into the lumen 5, or the like, and also functions as a grip portion when the catheter 1 is operated. A constituent material from which the hub 3 may be fabricated is not particularly limited, and for example, a thermoplastic resin such as polycarbonate, polyamide, polysulfone, polyarylate, or a methacrylate-butylene-styrene copolymer can be suitably used.

The strain relief 4 is made of an elastic material that surrounds the outer periphery of the shaft 2, and prevents kinking of the shaft 2 at the connection portion between the shaft 2 and the hub 3. As a constituent material from which the strain relief 4 may be fabricated, for example, natural rubber, silicone resin, or the like can be suitably used.

Next, a method for manufacturing the catheter 1 according to the present embodiment will be described.

First, a long or elongated core wire having an outer diameter equal to the inner diameter of the inner layer 10 is prepared. Next, the inner layer 10 is formed on the core wire. The inner layer 10 may be formed by extrusion molding, or may be formed by dip molding. Alternatively, the core wire may be inserted into the inner layer 10, which is configured as the tubular body.

Thereafter, the reinforcement body 20 is formed in a manner of covering at least a part of the inner layer 10. The reinforcement body 20 is formed by continuously winding the plurality of wires 21 on the inner layer 10 using a braid machine (braider).

Next, the marker 30 is disposed on the outer side of the inner layer 10 and the reinforcement body 20, and the marker 30 is crimped by a crimping machine to bite into the reinforcement body 20. Accordingly, the reinforcement body 20 receives a force from the outer side in the radial direction and is reduced in diameter, and the small (smaller) diameter portion 22 is formed. The small diameter portion 22 may not be formed in the marker 30. Thereafter, the outer layer 40 including the outer layer convex portion 42 is formed on the outer side of the inner layer 10, the reinforcement body 20, and the marker 30. Accordingly, the outer diameter difference (D1-D2), which is the difference between the outer diameter D1 of the shaft 2 in the portion where the marker 30 is disposed and the outer diameter D2 of the shaft 2 in the portion adjacent to the marker 30 in the axial direction X of the shaft 2, is smaller than twice the thickness T of the marker 30. In the present embodiment, the portion adjacent to the marker 30 in the axial direction X is a portion on the distal side and/or the proximal side of the marker 30. An inner diameter difference (D3-D4), which is a difference between an inner diameter D3 of the shaft 2 in the portion axially adjacent to the marker 30 in the axial direction X of the shaft 2 and an inner diameter D4 of the shaft 2 in the portion where the marker 30 is disposed, is smaller than twice the thickness T of the marker 30. The inner diameter D3 and the inner diameter D4 may be equal or may not be equal to each other. A method for forming the outer layer 40 is not particularly limited. For example, the outer layer 40 may be formed by the extrusion molding, or may be formed by the dip molding. Alternatively, the outer layer 40 may be formed by disposing the tubular body as a material of the outer layer 40 on the outer side of the inner layer 10, the reinforcement body 20, and the marker 30, and then heating the tubular body by covering the tubular body with a heat-shrinkable tube. The tubular body is softened or melted by heating, and is closely bonded to the outer side of the inner layer 10, the reinforcement body 20, and the marker 30 by a contraction force of the heat-shrinkable tube. Thereafter, the heat-shrinkable tube subjected to heat shrinkage is removed.

After the outer layer 40 is formed, the core wire is pulled out from the lumen 5 of the inner layer 10. Thereafter, the hub 3 and the strain relief 4 are attached to the shaft 2, and other members (for example, distal tip) are attached as necessary to complete the catheter 1.

As described above, the catheter 1 according to the present embodiment is configured to include the shaft 2 including the lumen 5 communicating or extending from the distal end to the proximal end. The shaft 2 includes the reinforcement body 20 including the plurality of wires 21 that are disposed in at least a part between the inner surface 11 of the shaft 2 forming the lumen 5 and the outer surface 41 of the shaft 2 and that are braided in the tubular shape, and at least one or more radiopaque markers 30 that are disposed between the reinforcement body 20 and the outer surface 41. The outer diameter difference (D1-D2), which is the difference between the outer diameter D1 of the shaft 2 in the portion where the marker 30 is disposed and the outer diameter D2 of the shaft 2 in the portion axially adjacent to the marker 30 in the axial direction X of the shaft 2, is smaller than twice the thickness T of the marker 30. Accordingly, the outer diameter of the shaft 2 provided with the reinforcement body 20 and the radiopaque marker 30 is not too large. Therefore, it is possible to prevent an increase in the thickness of the shaft 2 while disposing the marker 30 in the shaft 2 provided with the reinforcement body 20. By preventing the increase in the thickness of the shaft 2, the inner diameter of the shaft 2 can be made as large as possible while the outer diameter of the shaft 2 can be made as small as possible. Such a catheter 1 is particularly effective when a blood vessel (for example, the radial artery) to be introduced is thin and a blood vessel (for example, the artery of the lower limb) to be treated or diagnosed is thick.

The difference (D5-D6) between the maximum outer diameter D5 of the marker 30 and the maximum outer diameter D6 of the reinforcement body 20 in the portion axially adjacent to the marker 30 in the axial direction of the shaft 2 is smaller than twice the thickness T of the marker 30. Accordingly, the outer diameter of the shaft 3 provided with the reinforcement body 20 and the radiopaque marker 30 is not too large, and the inner diameter of the shaft 2 is not too small. Therefore, it is possible to prevent an increase in the thickness of the shaft 2 while disposing the marker 30 in the shaft 2 provided with the reinforcement body 20.

A thickness W1 of the outer layer 40 of the shaft 2 in the portion where the marker 30 is disposed and a thickness W2 of the outer layer 40 of the shaft 2 in the portion axially adjacent to the marker 30 in the axial direction X of the shaft 2 may be the same or W1 may be greater than W2. Accordingly, it is possible to prevent an increase in the thickness (outer dimension or outer diameter) of the shaft 2 while making the thicknesses W1 and W2 of the outer layer 40 uniform regardless of the presence or absence of the marker 30. If the thicknesses W1 and W2 of the outer layer 40 are sufficient, the outer layer convex portion 42 may not be formed (i.e., there may be no bulge) depending on the presence or absence of the marker 30. Therefore, although the thicknesses W1 and W2 of the outer layer 40 are thin and uniform, the outer diameter difference (D1-D2) may be smaller than the thickness of the marker 30.

The marker 30 includes at least one convex portion 32 on at least an inner surface side. Accordingly, the marker 30 is firmly fixed to the shaft 2. Therefore, detachment of the marker 30 from the shaft 2 can be prevented.

The effective length of the shaft 2 is 2100 mm or more. Accordingly, the catheter 1 can easily reach the artery of the lower limb from the artery of the arm.

The invention is not limited to the embodiment described above, and various modifications can be made by those skilled in the art within a scope of the technical idea of the invention. For example, in the embodiment described above, the number of markers 30 disposed in the shaft 2 is one, but may be two or more. The catheter 1 may be inserted from a blood vessel other than the artery of the arm. The catheter 1 may be used for the treatment or the diagnosis of the blood vessel other than the artery of the lower limb. The catheter 1 may be inserted into a bile duct, trachea, esophagus, urethra, or other biological lumens or a lumen in a living body and used for the treatment, the diagnosis, or the like.

In addition, according to another modification shown in FIG. 6, the inner diameter D4 of the shaft 2 in the portion where the marker 30 is disposed may be smaller than the inner diameter D3 of the axially adjacent portion 43 adjacent to the marker 30 in the axial direction X of the shaft 2. The inner diameter difference (D3-D4), which is the difference between the inner diameter D3 and the inner diameter D4, is preferably smaller than twice the thickness T of the marker 30. Accordingly, the inner diameter of the shaft 2 provided with the reinforcement body 20 and the radiopaque marker 30 is not too small. Therefore, it is possible to prevent an increase in the thickness of the shaft 2 while disposing the marker 30 in the shaft 2 provided with the reinforcement body 20. By preventing an increase in the thickness of the shaft 2, the inner diameter of the shaft 2 can be made as large as possible while the outer diameter of the shaft 2 can be made as small as possible. Such a catheter 1 is particularly effective when a blood vessel (for example, the radial artery) to be introduced is thin and a blood vessel (for example, the artery of the lower limb) to be treated or diagnosed is thick.

One of the outer diameter difference (D1-D2) and the inner diameter difference (D3-D4) alone may be smaller than twice the thickness T of the marker 30. Alternatively, both the outer diameter difference (D1-D2) and the inner diameter difference (D3-D4) may be smaller than twice the thickness T of the marker 30.

All of the difference (D5-D6) between the maximum outer diameter D5 of the marker 30 and the maximum outer diameter D6 of the reinforcement body 20 in the portion adjacent to the marker 30 in the axial direction of the shaft 2, the outer diameter difference (D1-D2) and the inner diameter difference (D3-D4) may be smaller than twice the thickness T of the marker 30. That is, all three of the noted differences, D5-D6, D1-D2 and D3-D4 may be smaller than twice the thickness T of the marker 30.

The detailed description above describes embodiments of a catheter and method of making a catheter representing examples of the inventive catheter and manufacturing method disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

### Reference Signs List

1 catheter
2 shaft
3 hub
4 strain relief
5 lumen
10 inner layer
11 inner surface
20 reinforcement body
21 wire
22 small diameter portion
30 marker
31 concave portion
32 convex portion
40 outer layer
41 outer surface
42 outer layer convex portion
43 adjacent portion
D1 outer diameter of outer layer convex portion
D2 outer diameter in portion adjacent to outer layer convex portion
D3 inner diameter of shaft in portion adjacent to marker
D4 inner diameter of shaft in portion where marker is disposed
D5 maximum outer diameter of marker
D6 outer diameter of portion adjacent to marker
T thickness of marker
X axis direction

What is claimed is:

1. A catheter comprising:
a shaft possessing a distal end and a proximal end, the shaft including a lumen surrounded by an inner surface of the shaft, the lumen extending from the distal end of the shaft to the proximal end of the shaft, the shaft also possessing an outer surface opposite the inner surface;
the shaft comprising:
a reinforcement body including a plurality of wires that are disposed at least at a part of the shaft between the inner surface of the shaft and the outer surface of the shaft, the plurality of wires being braided in a tubular shape; and
at least one radiopaque marker disposed between the reinforcement body and the outer surface of the shaft, the radiopaque marker possessing a thickness;
a difference between an outer diameter of the shaft in a portion where the radiopaque marker is disposed and an outer diameter of the shaft in a portion axially adjacent to the radiopaque marker in an axial direction of the shaft constituting an outer diameter difference, and a difference between an inner diameter of the shaft in the portion axially adjacent to the radiopaque marker in the axial direction of the shaft and an inner diameter of the shaft in the portion where the radiopaque marker is disposed constituting an inner diameter difference; and
at least one of the outer diameter difference and the inner diameter difference being smaller than twice the thickness of the radiopaque marker.

2. The catheter according to claim 1, wherein the shaft possesses a central axis, the radiopaque marker having a non-circular shape in a cross-section perpendicular to the central axis of the shaft.

3. The catheter according to claim 1, wherein the radiopaque marker possess an outer surface, the outer surface of the radiopaque marker including a plurality of ridges and grooves that alternate with one another in a circumferential direction of the radiopaque marker.

4. The catheter according to claim 1, wherein the radiopaque marker possess an outer surface, the outer surface of the radiopaque marker includes a plurality of concave portions and convex portions extending across a width-wise extent of the radiopaque marker and extending along a circumferential extent of the radiopaque marker.

5. The catheter according to claim 1, wherein the shaft comprises an inner layer and an outer layer.

6. The catheter according to claim 5, wherein the inner layer includes an inner surface that is the same as the inner surface of the shaft.

7. The catheter according to claim 6, wherein the outer layer includes an outer surface that is the same as the outer surface of the shaft.

8. The catheter according to claim 7, wherein the outer layer is made of a resin and the inner layer is made of a resin.

9. The catheter according to claim 5, wherein the outer layer includes an outer surface that is the same as the outer surface of the shaft.

10. The catheter according to claim 9, wherein the radiopaque marker is positioned radially outwardly of the inner layer.

11. The catheter according to claim 5, wherein the radiopaque marker is positioned radially outwardly of the inner layer.

12. The catheter according to claim 1, wherein the plurality of wires braided in the tubular shape form a tubular reinforcement body possessing a proximal end and a distal end, the radiopaque marker possessing a proximal end and a distal end, the proximal end of the tubular reinforcement body extending proximally beyond the proximal end of the radiopaque marker, the distal end of the tubular reinforcement body extending distally beyond the distal end of the radiopaque marker.

13. The catheter according to claim 1, wherein the radiopaque marker includes at least one convex portion on at least an inner surface side of the radiopaque marker.

14. The catheter according to claim 1, wherein the shaft has a length of 2100 mm or more.

15. A catheter comprising:
a shaft possessing a distal end and a proximal end, the shaft including a lumen extending from the distal end of the shaft to the proximal end of the shaft;
the shaft comprising:
a reinforcement body including a plurality of wires that are disposed at least at a part of the shaft between the inner surface of the shaft and the outer surface of the shaft, the plurality of wires being braided in a tubular shape; and
at least one radiopaque marker disposed between the reinforcement body and the outer surface of the shaft; and
a difference between a maximum outer diameter of the radiopaque marker and a maximum outer diameter of the reinforcement body in a portion axially adjacent to the radiopaque marker in an axial direction of the shaft is smaller than twice a thickness of the radiopaque marker.

16. The catheter according to claim 15, wherein the plurality of wires braided in the tubular shape form a tubular reinforcement body possessing a proximal end and a distal end, the radiopaque marker possessing a proximal end and a distal end, the proximal end of the tubular reinforcement body extending proximally beyond the proximal end of the radiopaque marker, the distal end of the tubular reinforcement body extending distally beyond the distal end of the radiopaque marker.

17. The catheter according to claim 15, wherein the radiopaque marker includes at least one convex portion on at least an inner surface side of the radiopaque marker.

18. The catheter according to claim 15, wherein the shaft has a length of 2100 mm or more.

19. The catheter according to claim 15, wherein the shaft comprises an inner layer and an outer layer, the radiopaque marker being located radially outwardly of the inner layer.

20. A catheter comprising:
a shaft possessing an open distal end and an open proximal end, the shaft including a lumen surrounded by an inner surface of the shaft, the lumen extending from the open distal end of the shaft to the open proximal end of the shaft, the shaft also possessing an outer surface opposite the inner surface;
the shaft comprising:
a reinforcement body including a plurality of wires braided in a tubular shape so that the reinforcement body is a tubular reinforcement body possessing opposite axial ends, the tubular reinforcement body being located radially between the inner surface of the shaft and the outer surface of the shaft, the tubular reinforcement body possessing an outer diameter, the tubular reinforcement body including an axially extending smaller diameter portion positioned axially between two axially extending portions of the tubular reinforcement body that are immediately axially adjacent the small diameter portion, the outer diameter of the smaller diameter portion of the tubular reinforcement body being less than the outer diameter of the two axially extending portions of the tubular reinforcement body that are immediately axially adjacent the smaller diameter portion; and
at least one radiopaque marker disposed radially between the reinforcement body and the outer surface of the shaft, the radiopaque marker being located in axially overlapping relation to the smaller diameter portion of the tubular reinforcement body, the radiopaque marker possessing a thickness;
a difference between an outer diameter of the shaft in a portion where the radiopaque marker is disposed and an outer diameter of the shaft in a portion axially adjacent to the radiopaque marker in an axial direction of the shaft constituting an outer diameter difference, and a difference between an inner diameter of the shaft in the portion axially adjacent to the radiopaque marker in the axial direction of the shaft and an inner diameter of the shaft in the portion where the radiopaque marker is disposed constituting an inner diameter difference; and
i) at least one of the outer diameter difference and the inner diameter difference being smaller than twice the thickness of the radiopaque marker, or ii) a difference between a maximum outer diameter of the radiopaque marker and a maximum outer diameter of the reinforcement body in a portion axially adjacent to the radiopaque marker in an axial direction of the shaft is smaller than twice a thickness of the radiopaque marker.
